(19) United States
(12) Patent Application Publication
(10) Pub. No.: US 2011/0072562 A1
(43) Pub. Date: Mar. 31, 2011

(54) Helmet bracket

(76) Inventor: Jonathon R. Prendergast, Newport Beach, CA (US)

(21) Appl. No.: 11/810,723

(22) Filed: Jun. 6, 2007

(51) Int. Cl. A42B 3/04; A61F 9/04; F16B 1/00; F41H 5/08

(52) U.S. Cl. 2/422; 2/426; 248/226.11; 89/36.05; 89/921; 89/926

(57) ABSTRACT

A headgear bracket for mounting night vision goggles includes a base having a headgear-facing surface. The headgear-facing surface includes a pocket defined by a pocket wall and a pair of flanges adapted to contact a headgear surface. The headgear bracket further includes an opening in the base adapted to receive an attachment means and a ballistic slug removably inserted into the pocket, the ballistic slug having an opening adapted to receive the attachment means. A surface of the ballistic slug is adapted to contact and conform substantially to the headgear surface.

### FIELD OF THE INVENTION

This invention in one or more embodiments relates to a headgear bracket, and more particularly to a helmet bracket for a mount for night vision goggles.

### BACKGROUND OF THE INVENTION

Night vision goggles are commonly used by military personnel for conducting operations in low light or night conditions. Assemblies for mounting night vision goggles to a helmet or other headgear are well known in the art. These mounting assemblies allow a user's hands to remain free while viewing a scene through the night vision goggles.

A mounting bracket adapted to receive a mount for night vision goggles may be attached to headgear. Often, the mounting bracket includes at least one opening through which an attachment means, such as a screw, is insertable to securely attach the mounting bracket to the headgear. Since the attachment means must also be inserted through the headgear, the headgear may also contain an opening to accommodate the attachment means.

The headgear worn by military personnel is designed for combat and is able to prevent a bullet shot from a pistol, such as a 9 mm, a .357 Magnum or an MP5 submachine gun, from penetrating the headgear. However, when bullets that would ordinarily be stopped and or sufficiently contained by the headgear encounter an opening in the helmet or the mounting bracket attachment means inserted through such opening, the force of the bullet impacts the weakened structural integrity of the headgear caused by the opening. As such, the bullet may penetrate the headgear and/or the attachment means may be pushed through the headgear and potentially into the wearer's head, posing serious danger to the wearer. It is desired to have a headgear bracket capable of preventing a bullet from penetrating the headgear and capable of preventing a mounting bracket attachment means from further penetrating the headgear and into a wearer's head.

Additionally, a hot shoe assembly may be mounted to headgear to provide an electrical connection to a battery pack or other power source for the night vision goggles. More specifically, the hot shoe assembly may be mounted to a hot shoe bracket which is then attached to headgear by attachment means similarly to the headgear bracket described above. Since an opening is created to allow the hot shoe bracket attachment means to penetrate the headgear, the portion of the helmet having the opening is vulnerable to being penetrated by a bullet. As such, it is desirable to provide a hot shoe bracket capable of preventing penetration by a bullet.

Moreover, it is generally desirable to provide a means to prevent a bullet or other ammunition from penetrating an opening or other structural weakness created in headgear, thus increasing safety for the headgear wearer.

### SUMMARY OF THE INVENTION

In one exemplary embodiment of the present invention, a headgear bracket for mounting night vision goggles is provided including a base having a headgear-facing surface. The headgear-facing surface may include a pocket defined by a pocket wall and a pair of flanges adapted to contact a headgear surface. The headgear bracket may further include an opening in the base adapted to receive an attachment means and a ballistic slug removably inserted into the pocket. The ballistic slug may have an opening adapted to receive the attachment means. A surface of the ballistic slug may be adapted to contact and conform substantially to the headgear surface.

In another exemplary embodiment of the present invention, a helmet on which night vision goggles are mountable is provided including an attachment means opening and a bracket having a bracket opening aligned with the attachment means opening. The bracket may include a helmet-facing surface having a pocket defined by a pocket wall and a ballistic slug removably inserted into the pocket. The ballistic slug may include a ballistic slug opening and a surface contacting and conforming substantially to a helmet surface. An attachment means may be inserted through the attachment means opening, the bracket opening and the ballistic slug opening to attach the bracket to the helmet.

In yet another exemplary embodiment of the present invention, a hot shoe bracket is provided including a base having a nut plate recess and a nut plate opening. At least one U-shaped member may extend from the base and is adapted to fit over a headgear rim. A nut plate is slidable within the nut plate recess and may protrude from the nut plate opening. A hot shoe assembly may include a block insulator having a plurality of electrical contacts and a hot shoe cover to cover the block insulator. A support plate may be located between the nut plate and the hot shoe assembly, the support plate having an opening through which the block insulator protrudes. The support plate may comprise titanium.

### BRIEF DESCRIPTION OF THE DRAWINGS

FIG. 1 is an orthogonal view of a helmet bracket attached to a helmet according to an exemplary embodiment of the present invention.

FIGS. 2 and 3 are front and rear orthogonal exploded views, respectively, of the helmet bracket of FIG. 1 including a ballistic slug and helmet attachment means.

FIG. 4 is a partial cross-sectional side exploded view of the helmet bracket of FIG. 1 as it would be attached to a helmet.

FIG. 5 is an orthogonal view of a ballistic slug according to an exemplary embodiment of the present invention.

FIG. 6 is an exploded view of a ballistic slug as it would be attached directly to a helmet according to an exemplary embodiment of the present invention.

FIG. 7 is a rear view of an exemplary headgear used in accordance with embodiments of the present invention having a rear opening.

FIGS. 8, 9 and 10 are an exploded view, a side view and a front view, respectively, of a hot shoe bracket in accordance with exemplary embodiments of the present invention.

FIG. 11 is an orthogonal view of a helmet bracket attached to a hot shoe bracket by a cable according to an exemplary embodiment of the present invention.

### DETAILED DESCRIPTION

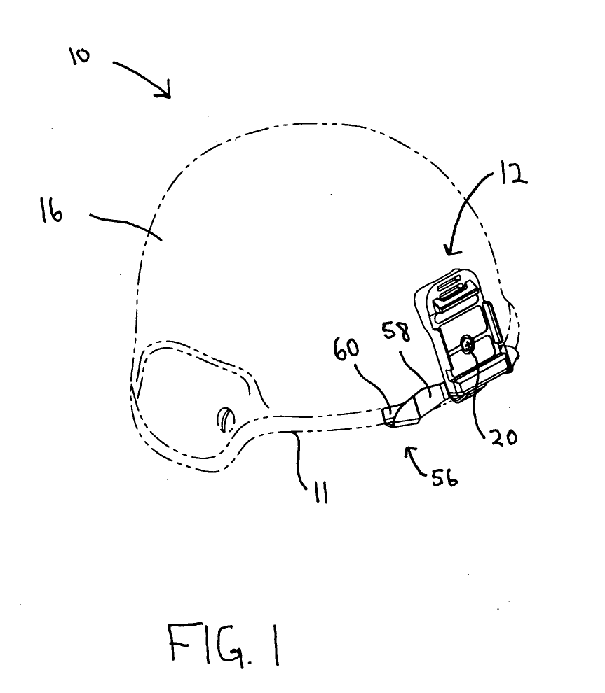

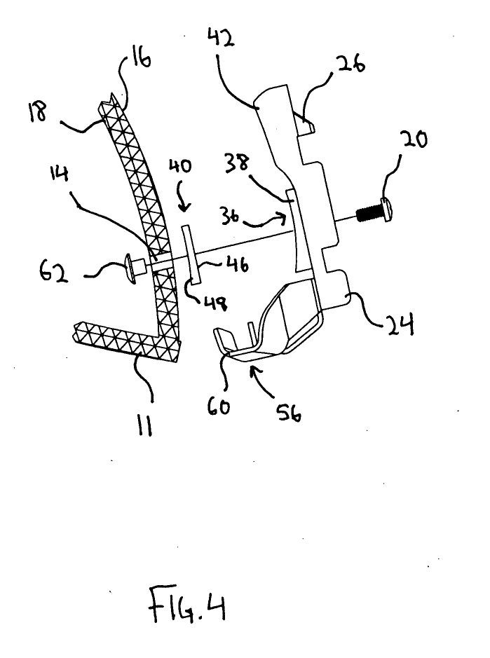

Referring to FIG. 1, headgear such as a helmet 10 is provided and adapted to be mounted with a helmet bracket 12. Although the headgear described herein will be a helmet commonly used by military personnel, it is understood that embodiments of the present invention may be incorporated into many different kinds of headgear. The helmet 10 defines an interior cavity shaped generally to conform to a wearer's head. More specifically, as also shown in FIG. 4, the helmet 10 includes an exterior surface 16, interior surface 18, and an opening 14 through which bracket attachment means, such as a screw 20, is insertable to attach the helmet bracket 12 to the helmet 10. Although the specifically described helmet 10 has only a single opening 14, it will be understood that the number of openings may vary depending on the number of attachment means desired to secure a bracket to the helmet.

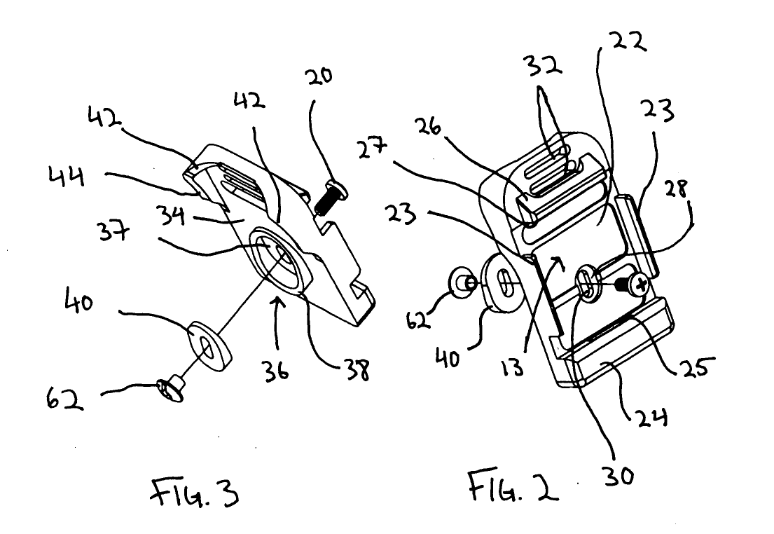

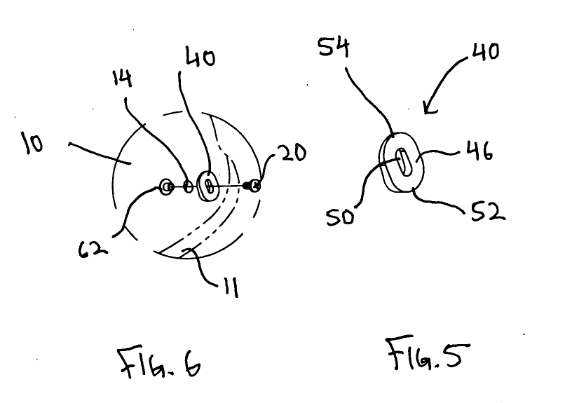

With reference now to FIG. 2, a base 13 of the helmet bracket 12 is adapted to receive a lock plate of a night vision goggle helmet mount (not shown). The base 13 of the helmet bracket 12 includes a lock plate receiving area 22 against which a lock plate inserted into the helmet bracket abuts. The lock plate receiving area 22 includes means to secure the lock plate against rotation comprising two side flanges 23, a foot 24 having a lower recess 25 into which a tongue of the lock plate is insertable, and a shoulder 26 including an upper recess 27 into which an upper lip of the lock plate snaps to secure the lock plate to the helmet bracket 12. The lock plate receiving area 22 further includes an opening 30 adapted to receive an attachment means and an attachment means recess 28 to permit the attachment means to be flush with or recessed from the receiving area. As such, attachment means 20 inserted into the opening 30 will not interfere with a lock plate attached to the receiving area 22. In one exemplary embodiment, the bracket opening 30 may have an area greater than a diameter of the helmet opening 14 (FIG. 5) to permit positional adjustment of the bracket 12 with respect to the helmet. For example, the bracket opening 30 may be substantially oval to permit adjustable vertical positioning of the helmet bracket 12. In one exemplary embodiment, the opening 30 has an area of from about 0.05 to about 0.25 square inch.

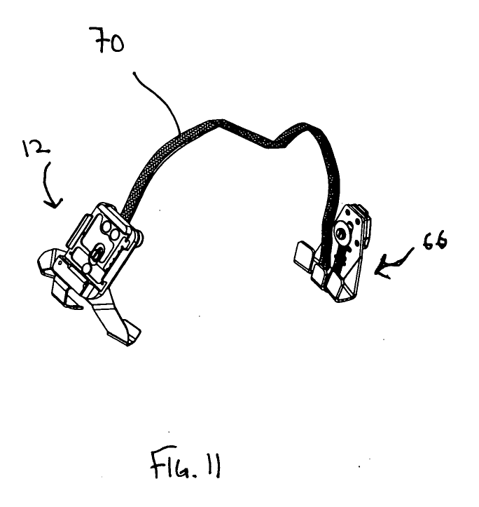

The helmet bracket 12 further contains a pair of strap or cable openings 32 above the shoulder 26 through which a strap or cable (FIG. 11) may be inserted to electrically connect the helmet bracket to a hot shoe as described in more detail below and/or to more securely attach the helmet bracket 12 to the helmet 10. Moreover, a clip 56 (FIG. 1) may be attached to the helmet bracket 12 to provide additional security against rotation of the helmet bracket 12 when the bracket is attached to a helmet 10 as described in more detail below. The clip 56 may comprise a pair of arms 58 having a generally U-shaped hand portion 60 which fits over a rim 11 of the helmet 10. Although a helmet bracket adapted to receive a lock plate of a night vision goggles mounting device is described herein, one of ordinary skill in the art will appreciate that other types of brackets and mounting devices may be used in conjunction with embodiments of the present invention.

Referring now to FIG. 3, a helmet-facing surface 34 of the helmet bracket 12 includes a ballistic slug pocket 36 defined by a flat base surface 37 and a pocket wall 38 protruding from the flat base surface. The ballistic slug pocket 36 is adapted to house a ballistic slug 40 as described in more detail below. In one exemplary embodiment, the pocket wall 38 surrounds the opening 30 in the helmet bracket 12 and a curvature of the pocket wall substantially matches the contour of the exterior surface 16 of the helmet 10. As such, the height of the pocket wall 38 nearer the foot 24 of the helmet bracket 12 may be greater than the height of the pocket wall nearer the shoulder 26 to account for the curvature of the helmet 10. For example a height of the pocket wall 38 may be about 0.05 inch at a first end and about 0.15 inch at a second end. However, a specific height of the pocket wall 38 is not critical, but rather the pocket wall should have a height to sufficiently and releasably secure a ballistic slug 40 inserted between the helmet 10 and the helmet bracket 12 as described in more detail below.

Additionally, a pair of support flanges 42 protrude from the helmet-facing surface 34 around a perimeter of the helmet facing surface 34 proximal the cable openings 32 to provide additional contact between the helmet bracket 12 and the helmet 10. The support flanges 42 include a contact portion 44 shaped to match substantially the contour of the helmet 10 which ensures a greater contact area between the helmet bracket 12 and the helmet. The support flanges 42 provide additional resistance to prevent a bullet from penetrating the helmet bracket opening 30 by absorbing the impact of the bullet and dispersing the energy of the impact over the surface of the helmet 10. Further, the support flanges 42 provide greater resistance to rotation and may prevent a portion of the helmet bracket 12 from fracturing due to force applied to a top half of the bracket. As shown in FIG. 3, the support flanges 42 taper back toward the helmet-facing surface 34 of the helmet bracket 12 at the top of the bracket to create an opening through which a strap or cable attached to the helmet bracket can extend.

With reference now also to FIG. 5, a ballistic slug 40 according to an exemplary embodiment of the present invention is adapted to be removably housed in the ballistic slug pocket 36 of the helmet bracket 12. The ballistic slug 40 comprises a relatively rigid material and includes a substantially flat lower surface 46 adapted to rest on the flat base surface 37 of the ballistic slug pocket 36. The ballistic slug 40 further includes a contoured helmet-facing surface 48 (FIG. 4) shaped to match substantially the contour of the helmet 10.

In one exemplary embodiment, a thickness of the ballistic slug 40 increasingly tapers from a first end 52 to a second end 54 while the helmet-facing surface 48 is slightly concave or bowl-shaped to correspond to the helmet surface. For example, a thickness of the first end 52 may be at least about 0.03 inch and a thickness of the second end 54 may be at least about 0.08 inch. In an alternate exemplary embodiment, the ballistic slug may have substantially parallel sides extending from a first end to a second end.

Further, when the ballistic slug 40 is inserted into the pocket 36, in one exemplary embodiment, the ballistic slug is flush with or protrudes slightly past the pocket wall 38. Accordingly, when the helmet bracket 12 is mounted onto the helmet 10, the ballistic slug 40 makes contact with the exterior surface 16 of the helmet 10. The contoured helmet-facing surface 48 allows maximum surface contact between the ballistic slug 40 and the helmet 10, allowing the slug to absorb as much impact as possible from a bullet and to distribute the force of the impact along the surface of the helmet.

The ballistic slug 40 includes an opening 50 generally corresponding to the attachment means opening 30 in the helmet bracket, the opening 50 adapted to receive an attachment means 20 as described in more detail below. In one exemplary embodiment, the opening 50 is generally oval to allow adjustment of the ballistic slug 40 with respect to a circular opening 14 on the helmet 10. However, it will be understood that the opening 50 may be of various shapes and sizes in accordance with embodiments of the present invention.

In one exemplary embodiment, the ballistic slug 40 is substantially oval. However, it will be understood by those of ordinary skill in the art that the ballistic slug 40 may be any one of a variety of shapes, such as, for example, circular, rectangular, or hexagonal. Additionally, the size, and therefore, the surface area, of the ballistic slug 40 may vary. In one exemplary embodiment, the surface area of the ballistic slug is about 0.32 square inch, but may be as small as about 0.20 square inch. It is noted that a thicker and/or larger ballistic slug 40 will generally be more effective than a smaller, thinner slug comprising the same material. However, due to the importance of weight considerations of equipment carried by military personnel, a lighter ballistic slug which effectively prevents penetration of a bullet may be more desirable than a heavier one.

In one exemplary embodiment, the ballistic slug 40 comprises a rigid material such as metal. The ballistic slug 40 may comprise, for example, aluminum, such as 6061 or 7075 aluminum, stainless steel, such as 17-4 heat treated steel, 300-series stainless steel or 12L14 soft steel, titanium, or various alloys or other combinations of metals. However, it will be understood by those of ordinary skill in the art that any sufficiently rigid or high tensile strength material may be used for the ballistic slug 40. For example, certain bullet resistant composite or woven materials may be used.

With reference now to FIGS. 1 and 4, the helmet bracket 12 may be attached to the helmet 10 as follows. A ballistic slug 40 may be inserted into the pocket 36 of the helmet bracket 12. Then, hand portions 60 of the clip 56 may be placed onto the rim 11 of the helmet 10, and the contact portion 44 of the support flanges 42 and the ballistic slug 40 may be rested against the exterior surface 16 of the helmet. Attachment means 20, such as a screw, is inserted through the openings 28, 50, 14 of the helmet bracket 12, the ballistic slug 40 and the helmet 14, respectively, and secured in place by, for example, a sex bolt 62. As will be understood by those of skill in the art, the attachment means are not limited to a screw and sex bolt, but may also be, for example, a rivet, nut and bolt, adhesive, VELCRO®, or the like. As such, if the attachment means 20 is impacted by a bullet fired at high velocity, the ballistic slug 40 will bear the force of impact and distribute such force along the surface of the helmet. Accordingly, the attachment means and the bullet will be prevented from further penetrating the helmet, and more importantly, will not penetrate the wearer's head.

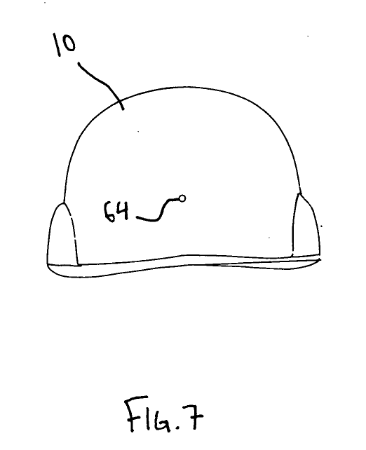

In an alternate embodiment of the present invention, as shown in FIG. 6, the ballistic slug 40 may be attached directly to the helmet 10 by attachment means 20 without an accompanying helmet bracket 12. As such, the ballistic slug will prevent a bullet from piercing an opening 14 in the helmet 10 even when a helmet bracket 12 is not mounted to the helmet. Additionally, the ballistic slug 40 may be used with various other types of mounting devices or other headgear components to reinforce a structurally vulnerable area. One of ordinary skill in the art will also appreciate that the ballistic slug 40 may be used with a helmet bracket 12 or other mounting device even if the ballistic slug 40 is separately attached to the helmet 10.

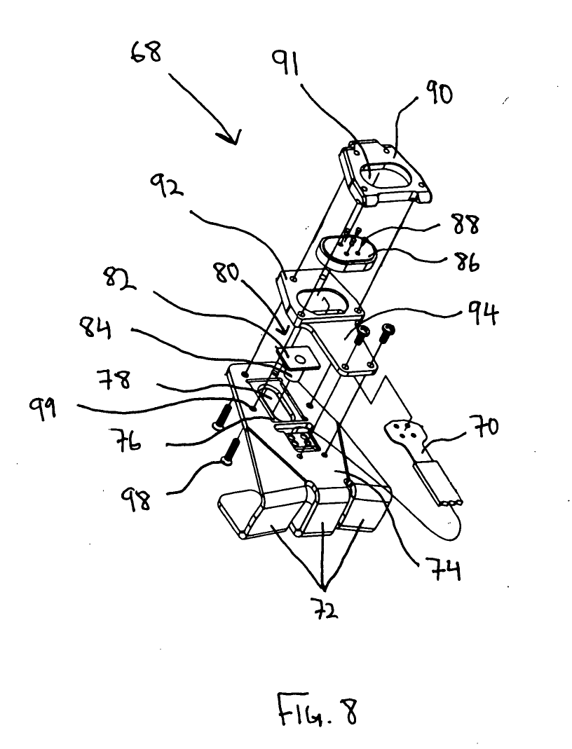

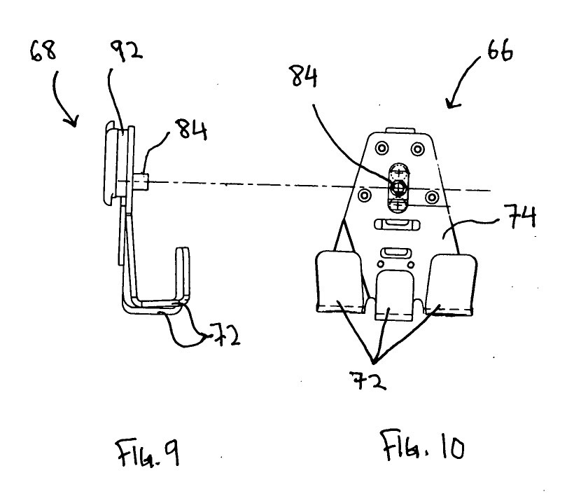

With reference now to FIG. 7, a helmet 10 may include a rear opening 64 adapted to receive an attachment member for a hot shoe bracket 66. With reference to FIGS. 8-10, a hot shoe bracket 66 is provided to attach a hot shoe assembly 68 to a helmet 10. The hot shoe assembly 68 is, in one exemplary embodiment, dove-tailed shaped and is adapted to provide an electrical connection between a battery pack (not shown) and a night vision goggles mount (not shown) through a cable 70 (FIG. 11) and the helmet bracket 12.

The hot shoe bracket 66 may comprise a plurality of feet 72 having a substantially U-shaped cross-section adapted to secure the hot shoe bracket 66 to the helmet rim 11. As shown in FIG. 9, the feet 72 may extend from a bracket base 74 at varying angles to account for curvature of the helmet 10. As will be appreciated by one of ordinary skill in the art, a continuous curved portion having a U-shaped cross-section “foot” would accomplish substantially the same function as the plurality of feet. The base 74 comprises a nut plate recess 76 and a nut plate opening 78 adapted to house a floating nut plate 80. More specifically, the floating nut plate 80 comprises a nut plate base 82 adapted to slide along the recess 76 and a threaded protrusion 84 extending through the nut plate opening 78. The threaded protrusion 84 may extend into the rear opening 64 of the helmet and an attachment means, such as a screw (not shown), may be attached to the threaded protrusion 84 from the interior surface 18 of the helmet to attach the hot shoe bracket 66 to the helmet 10.

A block insulator 86 comprising a plurality of contacts 88 and covered by a hot shoe cover 90 having a block insulator opening 91 may abut the nut plate base 82 to slidably secure the floating nut plate 80 within the nut plate recess 76 while allowing the block insulator 86 and contacts 88 to remain exposed. In one exemplary embodiment, the contacts 88 are electrically connected to the cable 70 which extends to the helmet bracket 12.

A support plate 92 may be attached between the hot shoe bracket 66 and the hot shoe cover 90, the support plate having a tail section 94 extending along part of the bracket base 74. The support plate 92 may include a hot shoe opening 96 corresponding to the opening 91 on the hot shoe cover 90 through which the block insulator 86 protrudes. Hot shoe attachment means 98, such as screws, may be inserted through attachment holes 99 in the hot shoe cover 90, support plate 92 and hot shoe bracket 66 to attach the components together.

Similarly to the helmet bracket 12 described above, the nut plate opening 78 and the rear opening 64 in the helmet create a more vulnerable region of the helmet 10 which may be susceptible to penetration by a bullet. Accordingly, in one exemplary embodiment, the support plate 92 and the nut plate base 82 are made from a rigid or high tensile strength material of a sufficient thickness to prevent penetration by a bullet. For example, the support plate 92 and the nut plate base 82 may be made from titanium, and specifically, Grade 5 titanium. In one exemplary embodiment, the thickness of the support plate 92 is at least about 0.06 inch and the thickness of the nut plate base 82 is at least about 0.05 inch.

As shown in FIG. 11, the cable 70 may be used to electrically connect the hot shoe bracket 66 to the helmet bracket 12. As noted above, the cable 70 may be inserted through cable openings 32 in the helmet bracket 12.

Although the present invention has been described through the use of exemplary embodiments, it will be appreciated by those of skill in the art that various modifications may be made to the described embodiments that fall within the scope and spirit of the invention as defined by the claims and their equivalents appended hereto.

What is claimed is:

1. A headgear bracket for mounting night vision goggles comprising:
a base comprising a headgear-facing surface, the headgear-facing surface comprising:
a pocket defined by a pocket wall; and
a pair of flanges adapted to contact a headgear surface;
an opening in the base adapted to receive an attachment means; and
a ballistic slug removably inserted into the pocket, the ballistic slug comprising an opening adapted to receive the attachment means;
wherein a surface of the ballistic slug is adapted to contact and conform substantially to the headgear surface.

2. The headgear bracket of claim 1, wherein the ballistic slug comprises a relatively rigid material.

3. The headgear bracket of claim 1, wherein the ballistic slug comprises a high tensile strength material.

4. The headgear bracket of claim 1, wherein the pocket wall conforms substantially to the headgear surface.

5. The headgear bracket of claim 1, wherein the ballistic slug has a surface area of at least about 0.2 square inch.

6. The headgear bracket of claim 1, wherein the attachment means is a screw and bolt.

7. The headgear bracket of claim 1, wherein each flange comprises a contact area conforming substantially to the headgear surface.

8. A helmet on which night vision goggles are mountable comprising:
an attachment means opening;
a bracket having a bracket opening aligned with the attachment means opening, the bracket having a helmet-facing surface comprising:
a pocket defined by a pocket wall; and
a ballistic slug removably inserted into the pocket, the ballistic slug including a ballistic slug opening and a surface contacting and conforming substantially to a helmet surface; and
an attachment means inserted through the attachment means opening, the bracket opening and the ballistic slug opening to attach the bracket to the helmet.

9. The helmet of claim 8, wherein the pocket wall conforms substantially to the headgear surface.

10. The helmet of claim 8, wherein the ballistic slug has a surface area of at least about 0.2 square inch.

11. The helmet of claim 8, wherein the attachment means is a screw and bolt.

12. The helmet of claim 8, wherein the bracket further comprises a pair of flanges adapted to contact and conform substantially to the helmet surface.

13. A ballistic slug mountable on headgear comprising:
a body including a headgear-facing surface; and
an opening in the body;
wherein the headgear-facing surface conforms substantially to a headgear surface.

14. The ballistic slug of claim 13, wherein a surface area of the ballistic slug is about 0.2 square inch.

15. The ballistic slug of claim 13, wherein the opening is adapted to receive an attachment means.

16. The ballistic slug of claim 15, wherein the attachment means is a screw and a bolt.

17. The ballistic slug of claim 13, further comprising a flat base surface.

18. The ballistic slug of claim 13, wherein the ballistic slug comprises a high tensile strength material.

19. The ballistic slug of claim 13, wherein the ballistic slug is substantially oval.

20. A hot shoe bracket comprising:
a base comprising a nut plate recess and a nut plate opening;
at least one U-shaped member extending from the base adapted to fit over a headgear rim;
a nut plate slidable within the nut plate recess and protruding from the nut plate opening;
a hot shoe assembly comprising:
a block insulator having a plurality of electrical contacts; and
a hot shoe cover to cover the block insulator; and
a support plate between the nut plate and the hot shoe assembly, the support plate comprising an opening through which the block insulator protrudes;
wherein the support plate and the nut plate comprise titanium.
